(19) United States
(12) Patent Application Publication
(10) Pub. No.: US 2026/0014545 A1
(43) Pub. Date: Jan. 15, 2026

(54) METHODS OF PREPARING METAL-ORGANIC FRAMEWORK COMPOUNDS

(71) Applicant: GENERAL ELECTRIC TECHNOLOGY GMBH, Baden (CH)

(72) Inventors: Jingjing YANG, Niskayuna, NY (US); Alexandra ANTONIO, Niskayuna, NY (US); Robert E. COLBORN, Niskayuna, NY (US); David MOORE, Niskayuna, NY (US); Mark DOHERTY, Niskayuna, NY (US)

(21) Appl. No.: 19/104,021

(22) PCT Filed: Dec. 22, 2022
(86) PCT No.: PCT/US2022/082243

§ 371 Date: Feb. 14, 2025

Related U.S. Application Data
(60) Provisional application No. 63/399,251, filed on Aug. 19, 2022.

(51) Int. Cl. B01J 20/22; B01D 53/02; B01J 20/28; C07F 3/02

(52) U.S. Cl. CPC B01J 20/226; B01D 53/02; B01J 20/28066; B01J 20/28076; C07F 3/02; B01D 2253/204; B01D 2253/306; B01D 2253/311; B01D 2257/504; B01D 2258/06

(57) ABSTRACT

Described herein is a method of preparing a metal-organic framework (MOF) compound including a MOF metal and a MOF linker. The method includes forming a mixture including a MOF metal precursor, a MOF linker precursor, a solvent, and optionally a base. The method also includes reacting the mixture under a reaction condition selected from the group consisting of an ambient reaction pressure, a mixture temperature less than about a boiling point of the solvent, an aqueous reaction mixture, and combinations thereof. Also described herein is a MOF compound prepared according to the method.

### RELATED APPLICATIONS

This application claims priority to U.S. Provisional Application Ser. No. 63/399,251, filed on Aug. 19, 2022, the contents of which are hereby incorporated by reference in their entirety.

### BACKGROUND

The field of the disclosure relates generally to methods of preparing metal-organic framework (MOF) compounds and MOF compounds produced by same, and more particularly to facile syntheses of MOF compounds.

MOF compounds are useful for a wide variety of purposes. For example, they are particularly useful in carbon capture sorbent systems, such as for use in post-combustion and direct air capture of CO₂.

MOF compounds are often produced via solvothermal processes. For example, one known solvothermal process includes reacting a MOF metal precursor and a MOF linker precursor in dimethylformamide (DMF) and methanol (MeOH) or ethanol (EtOH) in a sealed container at temperatures exceeding the boiling point of one of the solvents (e.g. 120° C.) under pressure, and then subsequently washing the product with methanol. However, such solvothermal processes present safety, toxicity, scalability, and cost challenges associated with the particular solvents used and the requirement for a reaction vessel that is capable of being exposed to elevated pressures and temperatures. Furthermore, some MOF compounds produced according to these solvothermal processes, such as Mg₂(dobpdc), are known to be unstable in the presence of humidity and/or water under various conditions. For example, as demonstrated by Wang, et. al., ACS Appl. Mater. Interfaces, 2021, 13, 17517, Mg₂(dobpdc) prepared according to known solvothermal procedures in DMF/methanol at 120° C. in a sealed vessel at elevated pressure exhibits new reflections and a 28% increase in the full width at half maximum (FWHM) of powder x-ray diffraction (PXRD) reflections upon exposure to DI water. This increase FWHM is indicative of partial dissolution or amorphization of the MOF framework. As another example, as demonstrated by Vitillo, et al, Mater. Chem. Front., 2017, 1, 444, Mg₂(dobpdc) was found to lose >80% of surface area after being stored in humid air with a humidity of >86% for 24 hours. Similarly, as demonstrated by Schoenecker, et. al., Ind. Eng. Chem. Red., 2012, 51, 6513, the related Mg₂(dobdc) material prepared via the known solvothermal method exhibits an 83% decrease in BET surface area after exposure to 80% relative humidity (RH) and reactivation. Furthermore, PXRD analysis of this material showed a slight loss of crystallinity, which when combined with the decreased BET surface area indicates structural degradation. Therefore, opportunities for pursuing alternative, facile reaction schemes are limited. Accordingly, there is a need for facile methods of preparing MOF compounds.

### BRIEF DESCRIPTION

In one aspect, provided herein is a method of preparing a metal-organic framework (MOF) compound including a MOF metal and a MOF linker, the method including: I) forming a mixture including: a MOF metal precursor, a MOF linker precursor, a solvent, and optionally a base; and II) reacting the mixture under a reaction condition selected from the group consisting of an ambient reaction pressure, a mixture temperature less than about a boiling point of the solvent, an aqueous reaction mixture, and combinations thereof.

In another aspect, provided herein is a metal-organic framework (MOF) compound including a MOF metal and a MOF linker, wherein the MOF compound is produced according to a method including: I) forming a mixture including: a MOF metal precursor, a MOF linker precursor, a solvent, and optionally a base; and II) reacting the mixture under a reaction condition selected from the group consisting of an ambient reaction pressure, a mixture temperature less than about a boiling point of the solvent, an aqueous reaction mixture, and combinations thereof.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is an exemplary method flow chart in accordance with the present disclosure;

FIG. 2 is a first representative reaction in accordance with the present disclosure; and

FIG. 3 is a second representative reaction in accordance with the present disclosure,

FIG. 4 is a third representative reaction in accordance with the present disclosure.

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION

The embodiments described herein overcome at least some of the disadvantages of known methods of preparing MOF compounds and known MOF compounds. The exemplary embodiments described herein include a method of preparing a metal-organic framework (MOF) compound including a MOF metal and a MOF linker, wherein the method includes: I) forming a mixture comprising: a MOF metal precursor, a MOF linker precursor, a solvent, and optionally a base; and II) reacting the mixture under a reaction condition selected from the group consisting of an ambient reaction pressure, a mixture temperature less than about a boiling point of the solvent, an aqueous reaction mixture, and combinations thereof. The method embodiments described herein use less toxic solvents, lower pressure, and/or require lower reaction temperatures as compared to known methods. Therefore, the exemplary embodiments present scalability, safety, cost, and waste-handling advantages as compared to at least some known methods of preparing MOF compounds.

The embodiments also include a metal-organic framework (MOF) compound including a MOF metal and a MOF linker, wherein the MOF compound is produced according to a method including: I) forming a mixture including: a MOF metal precursor, a MOF linker precursor, a solvent, and optionally a base; and II) reacting the mixture under a reaction condition selected from the group consisting of an ambient reaction pressure, a mixture temperature less than about a boiling point of the solvent, an aqueous reaction mixture, and combinations thereof. The MOF compound embodiments described herein present improved properties compared to the same MOF compounds prepared according to known methods. These improved properties include higher guest adsorption and/or higher surface area and/or higher pore volume as compared to at least some known MOF compounds prepared using known methods.

The MOF compounds described herein generally are easier to purify, e.g. via solvent washing, than the same MOF compounds prepared according to known methods. This improved purification stems from the relative ease of removing solvent molecules (e.g., water) from the MOF compounds described herein as compared to the removal of strongly bound solvent molecules (e.g., DMF) utilized to prepare the same MOF compounds according to known methods. In addition, the purified MOF compounds do not include strongly bound solvent molecules that decrease gas uptake, surface area, and/or overall pore volumes. Finally, the purification is improved by requiring less toxic purification methods.

The advantages and improved properties described herein are surprising in view of the known instability of the known MOF compounds in water when these compounds are produced by known methods.

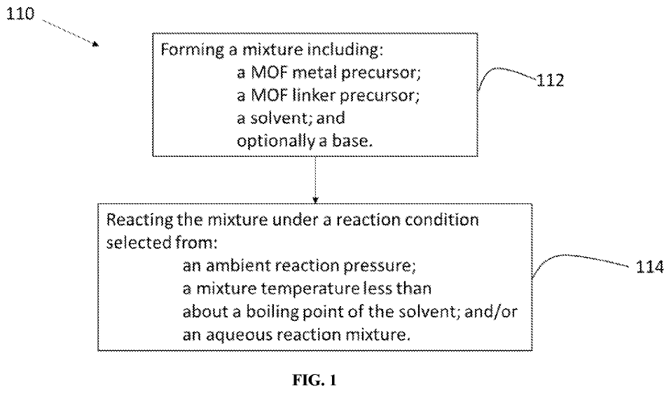

FIG. 1 is an exemplary method flow chart 110. In this exemplary embodiment, method flow chart 110 depicts the essential method steps of the method embodiments described herein and is not intended to limit the method embodiments. Method step 112 includes forming a mixture including: a MOF metal precursor; a MOF linker precursor; a solvent; and optionally a base. Method step 114 includes reacting the mixture under a reaction condition selected from: an ambient reaction pressure, a mixture temperature less than about a boiling point of the solvent, and/or an aqueous reaction mixture.

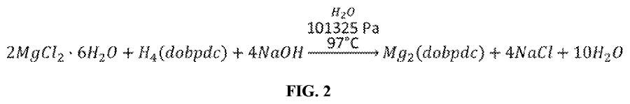

FIG. 2 is a first representative reaction in accordance with the present disclosure. In this exemplary embodiment, water is used as a solvent.

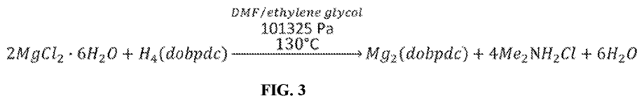

FIG. 3 is a second representative reaction in accordance with the present disclosure. In this exemplary embodiment, a mixture of DMF and ethylene glycol is used as a solvent.

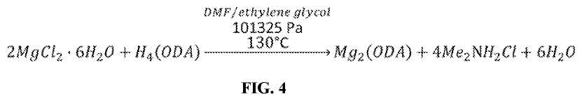

FIG. 4 is a third representative reaction in accordance with the present disclosure. In this exemplary embodiment, a mixture of DMF and ethylene glycol is used as a solvent.

In some embodiments, the MOF metal precursor may be any suitable MOF metal precursor known in the art that facilitates the method described herein. In other embodiments, the MOF metal precursor is a metal selected from the group consisting of alkali metals, alkaline earth metals, transition metals, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof. In some embodiments, the MOF metal precursor includes Mg. In some embodiments, the MOF metal precursor is Mg(NO₃)₂. In some embodiments, the MOF metal precursor is MgBr₂.

In some embodiments, the MOF linker precursor may be any suitable MOF linker precursor known in the art that facilitates the method described herein. In at least some embodiments, the MOF linker precursor is a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), protonated, partially and fully deprotonated forms thereof, and combinations thereof.

In the exemplary embodiment, the MOF linker precursor is 4,4′-dihydroxy-[1,1′-biphenyl]-3,3′-dicarboxylic acid (H₄dobpdc) and/or 4,4′-dioxidobiphenyl-3,3′-dicarboxylate (dobpdc⁴⁻). In some embodiments, dobpdc includes 4,4′-dihydroxy-[1,1′-biphenyl]-3,3′-dicarboxylic acid, its mono-carboxylate form, its di-carboxylate form, its mono-phenoxide form, its di-phenoxide form, and combinations thereof.

In some embodiments, the MOF linker precursor is one or more of the following linkers:

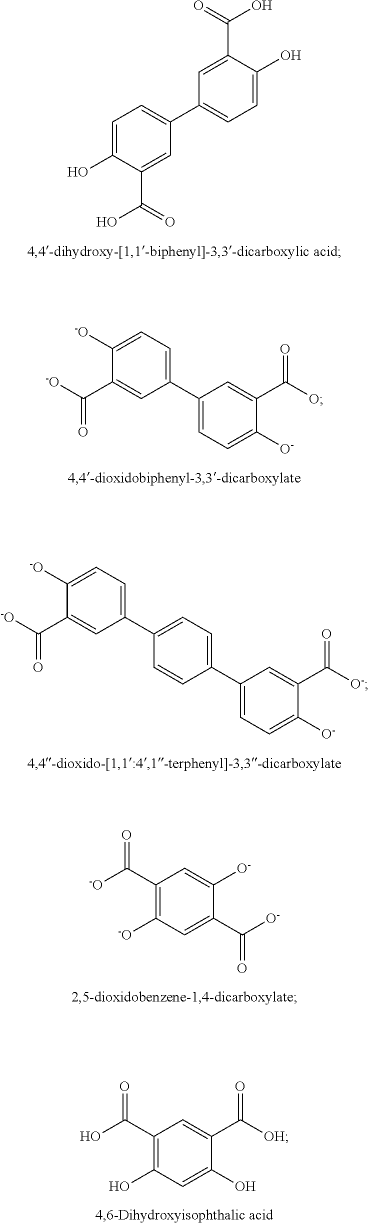

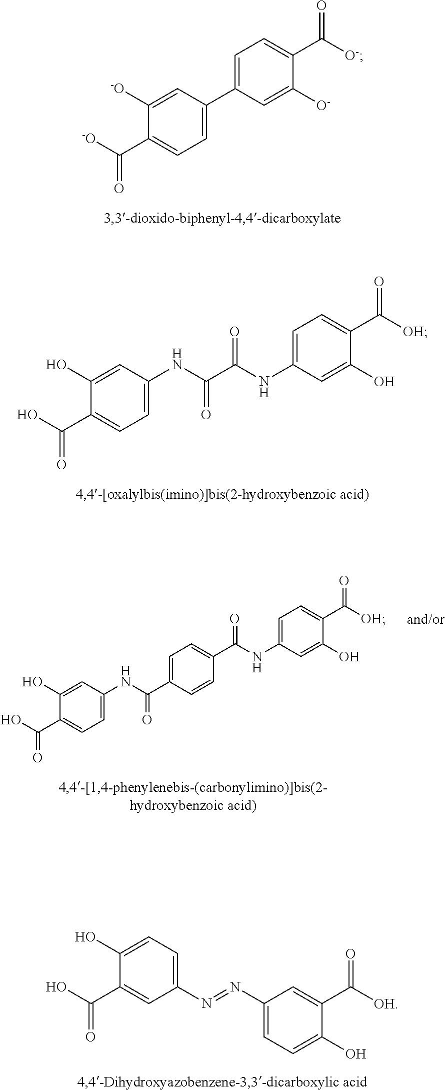

In many embodiments, the solvent may be any suitable solvent known in the art that facilitates the method described herein. In some embodiments, the solvent is a solvent selected from the group consisting of aqueous solvents, organic solvents, and combinations thereof. In some embodiments, the solvent is a solvent selected from the group consisting of water, dimethylformamide, ethylene glycol, ethanol, methanol, propanol, isopropanol, and combinations thereof.

In some embodiments, the solvent is an aqueous solvent. In some embodiments, the solvent is water. In some embodiments, the solvent is a water mixture.

In many embodiments, the base may be any suitable base known in the art that facilitates the method described herein. In some embodiments, the base is a base selected from the group consisting of strong bases, weak bases, inorganic bases, hydroxides, sodium hydroxide, potassium hydroxide, lithium hydroxide, cesium hydroxide, magnesium hydroxide, calcium hydroxide, organic bases, amines, and combinations thereof. In some embodiments, the base is sodium hydroxide.

In many embodiments, the reaction conditions may include any facile reaction conditions known in the art that facilitates the method described herein. In many embodiments, the reaction conditions do not include solvothermal reaction conditions. In many embodiments, the reaction conditions include a reaction condition selected from the group consisting of an ambient reaction pressure, a mixture temperature less than about a boiling point of the solvent, an aqueous reaction mixture, and combinations thereof.

In some embodiments, the reaction conditions include an ambient reaction pressure. In some embodiments, reacting the mixture includes reacting the mixture in an open reaction vessel. In some embodiments, reacting the mixture includes reacting the mixture in the absence of added pressure. In some embodiments, when the mixture is reacted, reacting does not occur in a closed container.

In some embodiments, the reaction conditions include ensuring a mixture temperature is less than about a boiling point of the solvent. In some embodiments, the reaction conditions include ensuring a mixture temperature is less than about 200° C. In some embodiments, the reaction conditions include ensuring a mixture temperature is less than about 190° C. In some embodiments, the reaction conditions include ensuring a mixture temperature is less than about 180° C. In some embodiments, the reaction conditions include ensuring a mixture temperature is less than about 170° C. In some embodiments, the reaction conditions include ensuring a mixture temperature is less than about 160° C. In some embodiments, the reaction conditions include ensuring a mixture temperature is less than about 150° C. In some embodiments, the reaction conditions include ensuring a mixture temperature is less than about 140° C. In some embodiments, the reaction conditions include ensuring a mixture temperature is less than about 130° C. In some embodiments, the reaction conditions include ensuring a mixture temperature is less than about 120° C. In some embodiments, the reaction conditions include ensuring a mixture temperature is less than about 115° C. In some embodiments, the reaction conditions include ensuring a mixture temperature is less than about 110° C. In some embodiments, the reaction conditions include ensuring a mixture temperature is less than about 105° C. In some embodiments, the reaction conditions include ensuring a mixture temperature is less than about 100° C. In some embodiments, the reaction conditions use a mixture temperature of about 100° C. In some embodiments, the reaction conditions use a mixture temperature of about 97° C. In some embodiments, the reaction conditions utilize a mixture temperature in a range of from about 80° C. to about 100° C. In some embodiments, the method includes heating the mixture under reflux. In some embodiments, the method includes heating the mixture under gentle reflux.

In many embodiments, reacting the mixture may occur for any suitable amount of time known in the art that facilitates the method described herein. In some embodiments, the mixture is reacted for an elapsed time in a range of from about 1 hour to about 72 hours. In some embodiments, the mixture is reacted for an elapsed time in a range of from about 1 hour to about 24 hours. In some embodiments, the mixture is reacted for an elapsed time in a range of from about 1 hour to about 12 hours. In some embodiments, the mixture is reacted for an elapsed time in a range of from about 1 hour to about 8 hours. In some embodiments, the mixture is reacted for an elapsed time in a range of from about 1 hour to about 4 hours. In some embodiments, the mixture is reacted for an elapsed time in a range of from about 1 hour to about 2 hours.

In many embodiments, the method may also include any further suitable processing steps known in the art that facilitate the success of the method described herein. Such processing steps include washing, drying, filtering, purifying, centrifuging, and any combinations thereof. In some embodiments, the method further includes washing the MOF compound. In some embodiments, the method also includes washing the MOF compound with a wash solvent selected from the group consisting of an alcohol, isopropanol, propanol, ethanol, methanol, water, and combinations thereof. In some embodiments, the method further includes washing the MOF compound with isopropanol.

In some embodiments, the MOF compound is a MOF compound of the MOF-74 family. In some embodiments, the MOF compound is Mg₂(dobpdc).

In many embodiments, the MOF compound possesses improved properties as compared to an identical MOF compound prepared according to a known method that facilitates the method described herein. In some embodiments, the MOF compound possesses a similar or higher guest adsorption and/or higher surface area and/or uptake as compared to an identical MOF compound prepared according to a known method. For example, Mg₂(dobpdc) prepared from known methods, such as in Milner, et. al., Chem. Sci., 2017, 160-174. Choe, et. al., Commun. Mater., 2021, 2, 3, and Kim et al., Science, 2020, 369, 392-396, exhibits N₂ uptake and BET surface area anywhere from 800-900 cm³/g and 3100-3300 m²/g, respectively, while Mg₂(dobpdc) prepared according to the method described herein exhibit ranges from 800-1000 cm³/g N₂ uptake and 3100-3400 m²/g BET surface area depending on the solvent combinations used.

In many embodiments, the MOF compound may be used according to any suitable purpose known in the art. In some embodiments, the MOF compound is used in a sorbent system. In some embodiments, the MOF compound is used in a carbon capture sorbent system. In some embodiments, the MOF compound is used in a moisture sorbent system. In some embodiments, the MOF compound is used for capturing a gas. In some embodiments, the MOF compound is used for post-combustion capturing of CO₂ and/or direct air capturing of CO₂.

Further aspects of the present disclosure are provided by the subject matter of the following clauses:
1. A method of preparing a metal-organic framework (MOF) compound comprising a MOF metal and a MOF linker, the method comprising: I) forming a mixture comprising: a MOF metal precursor, a MOF linker precursor, a solvent, and optionally a base; and II) reacting the mixture under a reaction condition selected from the group consisting of an ambient reaction pressure, a mixture temperature less than about a boiling point of the solvent, an aqueous reaction mixture, and combinations thereof.
2. The method in accordance with the preceding clause, wherein the MOF metal precursor comprises a metal selected from the group consisting of alkali metals, alkaline earth metals, transition metals, Mg, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof.
3. The method in accordance with any preceding clause, wherein the MOF linker precursor comprises a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), protonated, partially and fully deprotonated forms thereof, and combinations thereof.
4. The method in accordance with any preceding clause, wherein the solvent comprises a solvent selected from the group consisting of aqueous solvents, organic solvents, and combinations thereof.
5. The method in accordance with any preceding clause, wherein the solvent is an aqueous solvent.
6. The method in accordance with any preceding clause, wherein the solvent comprises a solvent selected from the group consisting of water, dimethylformamide, ethylene glycol, ethanol, methanol, propanol, isopropanol, and combinations thereof.
7. The method in accordance with any preceding clause, wherein the base comprises a base selected from the group consisting of strong bases, weak bases, inorganic bases, hydroxides, sodium hydroxide, potassium hydroxide, lithium hydroxide, cesium hydroxide, magnesium hydroxide, calcium hydroxide, organic bases, amines, and combinations thereof.
8 The method in accordance with any preceding clause, wherein reacting the mixture comprises reacting the mixture in an open reaction vessel.
9. The method in accordance with any preceding clause, wherein reacting the mixture comprises heating the mixture under reflux.
10. The method in accordance with any preceding clause, wherein the method further comprises washing the MOF compound with a wash solvent selected from the group consisting of an alcohol, isopropanol, propanol, ethanol, methanol, water, and combinations thereof.
11. A metal-organic framework (MOF) compound comprising a MOF metal and a MOF linker, wherein the MOF compound is produced according to a method comprising: I) forming a mixture comprising: a MOF metal precursor, a MOF linker precursor, a solvent, and optionally a base; and II) reacting the mixture under a reaction condition selected from the group consisting of an ambient reaction pressure, a mixture temperature less than about a boiling point of the solvent, an aqueous reaction mixture, and combinations thereof.
12. The MOF compound in accordance with any preceding clause, wherein the MOF metal precursor comprises a metal selected from the group consisting of alkali metals, alkaline earth metals, transition metals, Mg, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof.
13. The MOF compound in accordance with any preceding clause, wherein the MOF linker precursor comprises a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), protonated, partially and fully deprotonated forms thereof, and combinations thereof.
14. The MOF compound in accordance with any preceding clause, wherein the MOF compound is Mg₂(dobpdc).
15. The MOF compound in accordance with any preceding clause, wherein the solvent comprises a solvent selected from the group consisting of aqueous solvents, organic solvents, and combinations thereof.
16. The MOF compound in accordance with any preceding clause, wherein the solvent is an aqueous solvent.
17. The MOF compound in accordance with any preceding clause, wherein the solvent comprises a solvent selected from the group consisting of water, dimethylformamide, ethylene glycol, ethanol, methanol, propanol, isopropanol, and combinations thereof.
18. The MOF compound in accordance with any preceding clause, wherein the base comprises a base selected from the group consisting of strong bases, weak bases, inorganic bases, hydroxides, sodium hydroxide, potassium hydroxide, lithium hydroxide, cesium hydroxide, magnesium hydroxide, calcium hydroxide, organic bases, amines, and combinations thereof.
19. The MOF compound in accordance with any preceding clause, wherein reacting the mixture comprises reacting the mixture in an open reaction vessel.
20. The MOF compound in accordance with any preceding clause, wherein reacting the mixture comprises heating the mixture under reflux.
21. The MOF compound in accordance with any preceding clause, wherein the method further comprises washing the MOF compound with a wash solvent selected from the group consisting of an alcohol, isopropanol, propanol, ethanol, methanol, water, and combinations thereof.
22. A sorbent system comprising the MOF compound in accordance with any preceding clause.
23. A method of using the MOF compound in accordance with any preceding clause, the method comprising using the MOF compound for capturing a gas.
24. A method of using the MOF compound in accordance with any preceding clause, the method comprising using the MOF compound for post-combustion capturing of CO₂ and/or direct air capturing of CO₂.

References to “some embodiments” in the above description are not intended to be interpreted as excluding the existence of additional embodiments that also incorporate the recited features.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Preparation of Mg₂(Dobpdc) with an Aqueous Solvent Under Facile Reaction Conditions

Mg₂(dobpdc) was prepared according to the reaction scheme of FIG. 2 at a 2.5 L scale. First, sodium hydroxide (1 mol, 40.0 g) was dissolved in water (2.0 L) in an open flask. Next, H₄ (dobpdc) (0.250 mol, 68.5 g) was added via addition funnel to form a slurry which was sparged with N₂ for 1 hour. Simultaneously, MgCl₂ hexahydrate (0.562 mol, 114.25 g) was dissolved in 500 mL of water in a separate vessel and sparged with N₂ for 1 hour. The metal salt solution was added via addition funnel to the slurry to precipitate out MOF. The mixture was reacted in an open flask under ambient pressure and gentle reflux at about 97° C. for anywhere from 14-72 hours. The reaction produced MOF compound Mg₂(dobpdc), which was subsequently washed thrice with water and thrice with isopropanol and stored in alcohol. For surface area measurements, the material was washed with methanol to obtain the highest uptake. The yield was about 80 g of Mg₂(dobpdc) for each of three separate batches.

It was determined that the produced Mg₂(dobpdc) possessed improved properties (e.g. higher 77K N₂ gas adsorption, higher surface area, and higher pore volume) as compared to Mg₂(dobpdc) prepared in a DMF/EtOH solvent, which can exhibit an uptake ranging from 800-900 cm³/g, a BET (Langmuir) surface area ranging from 3100-3300 (3700-4000) m²/g, and a pore volume of 1.25 cm³/g. For instance, the Mg₂(dobpdc) prepared with an aqueous solvent exhibited an uptake of 1120 cm³/g, a BET (Langmuir) surface area of 3341 (5430) m²/g, and a pore volume of 1.73 cm³/g. To measure the isotherms, a Micromeritics ASAP 2020 gas adsorption analyzer was used. Samples were loaded into weighed analysis tubes, loaded with sample, capped with Transeals and moved to the degas station. They were activated under vacuum at various temperatures until the static outgas rate was less than 10 μmHg/min. After degassing, the tube was removed from the degas station under N₂ and weighed to determine the mass of the sample in the tube. For cryogenic N₂ measurements, an isothermal jacket was fitted on the tube. BET measurements using N₂ at 77K were performed on the samples and the surface area was calculated via the Micromeritics software.

### Example 2. Preparation of Mg₂(Dobpdc) with a Non-Aqueous Solvent Under Facile Reaction Conditions

Mg₂(dobpdc) was prepared according to the reaction scheme of FIG. 3 at a 5 g scale. MgCl₂ hexahydrate (6.0 g), H₄ (dobpdc) (4.0 g), and DMF/ethylene glycol (40 mL/40 mL) were combined in an open flask to form a mixture. The mixture was reacted in the open flask under ambient pressure and gently stirred at a temperature of about 130° C. for about 72 hours. The reaction produced MOF compound Mg₂(dobpdc), which was subsequently washed with DMF and ethanol and then dried. The yield was about 4.5 g of Mg₂(dobpdc).

It was determined that the produced Mg₂(dobpdc) possessed similar properties (e.g. similar 77K N₂ gas adsorption, similar surface area, and similar pore volume) as compared to Mg₂(dobpdc) prepared in a DMF/EtOH solvent, which can exhibit an uptake ranging from 800-900 cm³/g, a BET (Langmuir) surface area ranging from 3100-3300 (3700-4000) m²/g, and a pore volume of 1.25 cm³/g. For instance, the Mg₂(dobpdc) prepared in a DMF/ethylene glycol solvent exhibited an uptake of 780 cm³/g, a BET (Langmuir) surface area of 2070 (3428) m²/g, and a pore volume of 1.21 cm³/g. To measure the isotherms, a Micromeritics ASAP 2020 gas adsorption analyzer was used. Samples were loaded into weighed analysis tubes, loaded with sample, capped with Transeals and moved to the degas station. They were activated under vacuum at various temperatures until the static outgas rate was less than 10 μmHg/min. After degassing, the tube was removed from the degas station under N₂ and weighed to determine the mass of the sample in the tube. For cryogenic N₂ measurements, an isothermal jacket was fitted on the tube. BET measurement using N₂ at 77K were performed on the samples and the surface area was calculated via the Micromeritics software.

### Example 3. Preparation of Mg₂(ODA) with a Non-Aqueous Solvent Under Facile Reaction Conditions

Mg₂(ODA) was prepared according to the reaction scheme of FIG. 4 at a 5 g scale. MgCl₂ hexahydrate (4.066 g), H₄(ODA) (3.60 g), and DMF/ethylene glycol (40 mL/40 mL) were combined in an open flask to form a mixture. The mixture was reacted in the open flask under ambient pressure and gently stirred at a temperature of about 130° C. for about 72 hours. The reaction produced MOF compound Mg₂(ODA), which was subsequently washed with DMF and ethanol and then dried, the yield was about 4 g of Mg₂(ODA).

### Example 4. Physical Property Comparison

The physical properties of Mg₂(dobpdc) produced according to the present disclosure and Mg₂(dobpdc) produced according to conventional synthesis were determined and compared. The comparison is shown in the table below.

TABLE 1
Comparative physical properties of Mg₂(dobpdc) across synthetic routes.

| | | Activation | | | | | Measured | Calculated |
|---|---|---|---|---|---|---|---|---|
| Synthesis | Wash | Temperature | Uptake | Langmuir | BET | PSD | Pore Volume | Pore Volume |
| Conditions | Solvent(s) | (° C.) | (cm³/g) | (m²/g) | (m²/g) | (d_{p, peak} nm) | (cm³/g) | (cm³/g) |
| DMF/EtOH | DMF/ | 180 | 527 | 2347 | 1318 | N.D. | 0.8154 | 0.817 |
| 130° C. | EtOH | | | | | | | |
| DMF/EtOH | DMF/ | 250 | 519 | 1425 | 1065 | 1.9332 | 0.7296 | 0.805 |
| 130° C. | EtOH | | | | | | | |
| DMF/EG | DMF, | 180² | 781 | 3428 | 2070 | N.D. | 1.2076 | 1.211 |
| 130° C. | EtOH | | | | | | | |
| DMF/EG | DMF, | 250 | 1017 | 3863 | 2999 | 1.9918 | 1.3448 | 1.578 |
| 130° C. | EtOH | | | | | | | |
| H₂O | H₂O, | 180¹ | 1121 | 5039 | 2814 | N.D. | 1.7332 | 1.739 |
| 97° C. | MeOH | | | | | | | |
| H₂O | H₂O, IPA | 250 | 1097 | 4337 | 3481 | 1.9985 | 1.4707 | 1.702 |
| 97° C. | | | | | | | | |

N.D. = not determined
¹= same conditions as Example 1, but different sample
²= same conditions and sample as Example 2

Pore volume is readily calculated from 77 K N₂ uptake measurements by taking into account the density of liquid N₂ at 77 K (0.8064 g N₂/cm³), the molecular weight of N₂ (28.02 g/mol), and the molar gas constant of 22.4 L/mol. For example, for an observed N₂ uptake at 77 K of 1120 cm³/g, a pore volume of 1.737 cm³/g may be calculated as follows:

$$( 1120 \frac{{cm}^{3}}{ℊ} ) ( \frac{1 mol {N}_{2}}{22400 {cm}^{3}} ) ( \frac{28.02 ℊ {N}_{2}}{mol {N}_{2}} ) ( \frac{1}{0.8064} \frac{{cm}^{3}}{ℊ {N}_{2}} ) = 1.737 \frac{{cm}^{3}}{ℊ}$$

The data of Table 1 illustrate that the physical properties of Mg₂(dobpdc) produced according to the present disclosure meet or outperform the physical properties of Mg₂(dobpdc) produced according to conventional synthesis.

Unless otherwise indicated, approximating language, such as “generally,” “substantially,” and “about,” as used herein indicates that the term so modified may apply to only an approximate degree, as would be recognized by one of ordinary skill in the art, rather than to an absolute or perfect degree. Accordingly, a value modified by a term or terms such as “about,” “approximately,” and “substantially” is not to be limited to the precise value specified. In at least some instances, the approximating language may correspond to the precision of an instrument for measuring the value. Additionally, unless otherwise indicated, the terms “first,” “second,” etc. are used herein merely as labels, and are not intended to impose ordinal, positional, or hierarchical requirements on the items to which these terms refer. Moreover, reference to, for example, a “second” item does not require or preclude the existence of, for example, a “first” or lower-numbered item or a “third” or higher-numbered item.

Although specific features of various embodiments of the invention may be shown in some drawings and not in others, this is for convenience only. Moreover, references to “some embodiments” in the above description are not intended to be interpreted as excluding the existence of additional embodiments that also incorporate the recited features. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of preparing a metal-organic framework (MOF) compound comprising a MOF metal and a MOF linker, the method comprising:
I) forming a mixture comprising:
a MOF metal precursor;
a MOF linker precursor;
a solvent; and
optionally a base; and
II) reacting the mixture under a reaction condition selected from the group consisting of an ambient reaction pressure, a mixture temperature less than about a boiling point of the solvent, an aqueous reaction mixture, and combinations thereof.

2. The method in accordance with claim 1, wherein the MOF metal precursor comprises a metal selected from the group consisting of alkali metals, alkaline earth metals, transition metals, Mg, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof.

3. The method in accordance with claim 1, wherein the MOF linker precursor comprises a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), protonated, partially and fully deprotonated forms thereof, and combinations thereof.

4. The method in accordance with claim 1, wherein the solvent comprises a solvent selected from the group consisting of aqueous solvents, organic solvents, and combinations thereof.

5. The method in accordance with claim 1, wherein the solvent is an aqueous solvent.

6. The method in accordance with claim 1, wherein the solvent comprises a solvent selected from the group consisting of water, dimethylformamide, ethylene glycol, ethanol, methanol, propanol, isopropanol, and combinations thereof.

7. The method in accordance with claim 1, wherein the base comprises a base selected from the group consisting of strong bases, weak bases, inorganic bases, hydroxides, sodium hydroxide, potassium hydroxide, lithium hydroxide, cesium hydroxide, magnesium hydroxide, calcium hydroxide, organic bases, amines, and combinations thereof.

8. The method in accordance with claim 1, wherein reacting the mixture comprises reacting the mixture in an open reaction vessel.

9. The method in accordance with claim 1, wherein reacting the mixture comprises heating the mixture under reflux.

10. The method in accordance with claim 1, wherein the method further comprises washing the MOF compound with a wash solvent selected from the group consisting of an alcohol, isopropanol, propanol, ethanol, methanol, water, and combinations thereof.

11. A metal-organic framework (MOF) compound comprising a MOF metal and a MOF linker, wherein the MOF compound is produced according to a method comprising:
I) forming a mixture comprising:
a MOF metal precursor;
a MOF linker precursor;
a solvent; and
optionally a base; and
II) reacting the mixture under a reaction condition selected from the group consisting of an ambient reaction pressure, a mixture temperature less than about a boiling point of the solvent, an aqueous reaction mixture, and combinations thereof.

12. The MOF compound in accordance with claim 11, wherein the MOF metal precursor comprises a metal selected from the group consisting of alkali metals, alkaline earth metals, transition metals, Mg, Ca, Mn, Cr, Fe, Co, Ni, Cu, Zn, ions thereof, hydrates thereof, salts thereof, halides thereof, fluorides thereof, chlorides thereof, bromides thereof, iodides thereof, nitrates thereof, acetates thereof, sulfates thereof, phosphates thereof, carbonates thereof, oxides thereof, formates thereof, carboxylates thereof, and combinations thereof.

13. The MOF compound in accordance with claim 11, wherein the MOF linker precursor comprises a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), protonated, partially and fully deprotonated forms thereof, and combinations thereof.

14. The MOF compound in accordance with claim 11, wherein the MOF compound is Mg₂(dobpdc).

15. The MOF compound in accordance with claim 11, wherein the solvent comprises a solvent selected from the group consisting of aqueous solvents, organic solvents, and combinations thereof.

16. The MOF compound in accordance with claim 11, wherein the base comprises a base selected from the group consisting of strong bases, weak bases, inorganic bases, hydroxides, sodium hydroxide, potassium hydroxide, lithium hydroxide, cesium hydroxide, magnesium hydroxide, calcium hydroxide, organic bases, amines, and combinations thereof.

17. The MOF compound in accordance with claim 11, wherein reacting the mixture comprises reacting the mixture in an open reaction vessel.

18. A sorbent system comprising the MOF compound in accordance with claim 11.

19. A method of using the MOF compound in accordance with claim 11, the method comprising using the MOF compound for capturing a gas.

20. A method of using the MOF compound in accordance with claim 11, the method comprising using the MOF compound for post-combustion capturing of CO₂ and/or direct air capturing of CO₂.
